(19) United States
(12) Patent Application Publication
(10) Pub. No.: US 2006/0052283 A1
(43) Pub. Date: Mar. 9, 2006

(54) The Sperm Binding Ligand HIPRTY and Its Involvement in Embryo Cleavage

(76) Inventor: George Pieczenik, New York, NY (US)

Correspondence Address: DR. GEORGE PIECZENIK, APT. 1F, 412 EAST 55TH STREET, NEW YORK, NY, 10022, US

(21) Appl. No.: 10/711,268

(22) Filed: Sep. 7, 2004

(51) Int. Cl. A61K 38/08; C07K 7/06

(52) U.S. Cl. 514/2; 514/17; 530/330

(57) ABSTRACT

A sperm-binding ligand sequence with the amino acid sequence HIPRTY is claimed. Using proprietary bio-informatic analysis, this sperm-binding ligand sequence was shown to also precisely correspond with a previously characterized highly conserved protein-protein interaction site in the Axin protein at position 370 to 375. The Axin protein sequence HIPRTY is found within the binding site of Axin to a known sperm surface protein, Glycogen Synthase Kinase-3. This result not only provides proof of the utility of this technique to identify cell surface ligands in mammalian gametes, but it also provides the first molecular data supporting a potential role for spermatozoa in facilitating developmental axis formation in mammalian embryos. The HIPRTY peptide can be useful in fertility therapy and diagnosis.

Understanding cell-cell interactions is a key goal in many areas of cell and developmental biology. This is particularly so in the case of early development. Despite years of basic research into such interaction, much remains mysterious (Myles et al., 1994; Evans et al., 1995; McElhinny and Warner, 2000; Neganova et al., 2000; Ensslin and Shur, 2003). Elucidating the key components involved with cell-cell interactions in fertilization, cleavage, implantation and other phenomenon will not only provide great insights into the basic developmental process but will also be of great value in the development of interventional techniques in assisted reproduction and contraception. A variety of techniques have been used in analyzing the components involved with these cell-cell phenomena. For the most part these strategies have attempted to define the cell surface proteome and determine the function and importance of specific isolated proteins via genetic or biochemical analysis. Ideally, the vital cell surface could be screened in situ allowing for a more natural and complete understanding components involved in cell-cell interactions.

One approach is provided through the use of combinatorial libraries. (Cortese et al., 1994; Pasqualini and Ruoslahti, 1996; Szardenings et al., 1997; Pieczenik, 1999, Pieczenik 2003a, b). With the completion of the human genome sequence, the information for all the possible sequences involved in all interactions in the human is theoretically available. The question is how to sort out all the interactions. A peptide combinatorial library can contain all possible sequences in the range of 4-7 amino acids. The great preponderance of non-covalent binding interactions at the molecular level are made of interactions between small number of amino acids in the range of 4-7 or at extremes, multiples of such sizes. Another constraint is the number of unique peptide sequences in the genome. Even if the whole genome were coding i.e. 3 billion nucleotides coding for a unique 1 billion amino acids, then any given sequence could be uniquely specified by 6-7 amino acids. This can be seen in that 20 to the power 6-7 has a range of is 64 million to 1.28 billion. In reality, there are only about 20,000 coding sequences in the genome and even if we generously give each coding 500 amino acid lengths, then we are talking about 10 million unique sequences. This data base can therefore be uniquely specified or searched by 5-6 amino acids. That is, we would expect to see any given 6 amino acid sequence appear once in the human genome. The figure of 5 amino acids is considered the unique determinant for almost all known protein sequences. Therefore, both the physical chemistry of interactions and the global statistic of the genome codings suggest that 5 amino acids is a unique peptide and protein identifier (Pieczenik, 2003 b).

The following patents are incorporated by reference. The U.S. inventor Dr. George Pieczenik, U.S. Pat. No. 5,866,363 issued Feb. 2, 1999.

U.S. Pat. No. 6,605,448 to Dr. George Pieczenik issued on Aug. 12, 2003 is also incorporated by reference.

Therefore, the genome is effectively a closed informational circle for sequences in the range of 4-7 amino acids. The universe of antibody-antigen interactions is also not infinite but defined in size (Pieczenik, 2003b). This is a strategy that was used in sequencing the φX174 (Sanger et al, 1975). The 4×1 74 genome is actually a physical circle and therefore continuous random sequencing will effectively “close the circle”. A combinatorial library containing all possible 4-7 amino acid sequences essentially creates a “virtual” closed circle of all possible interacting ligands for the expressed genome or protein phenotype. This aspect of the closed and limited size of both the genome codings and the combinatorial libraries guarantee that if a binding ligand is identified in the range of 4-7 amino acids then chances are that it is unique to the function of that specific binding.

We have screened intact live mouse spermatozoa using a proprietary combinatorial peptide library selection protocol. Following the initial 4 rounds of binding, amplification and selection, a consistent set of peptide sequences emerged as a putative ligand interacting with sperm surface component. It was the HIPRTY amino acid sequence. Upon further analysis, this peptide sequence was shown to be a perfect match for a well characterized, completely conserved region of the Axin protein mediating interaction w/glycogen synthase kinase-3 (GSK-3). GSK-3 has been recently identified as a mammalian sperm surface protein with a potential role in the regulation of motility (Vijayaraghavan et al., 1996; Smith et al., 1999; Vijayaraghavan et al., 2000). This result demonstrates that whole cell combinatorial peptide library selection is a valid approach for the identification of sperm surface proteins. Furthermore, it provides the first molecular evidence supporting the theory that sperm may play a role in the initial determination of polarity in mouse embryos via a direct interaction with Axin.

Materials and Methods:

A mouse sperm suspension was isolated from cauda epididymides of fertile male CB6F1 mice. Briefly, the epididymides and vas deferens were dissected in a 35 mm dish containing 5 ml of CZB (pre-equilibrated at 37° C. and 5% CO2) and gently compressed to release sperm. Following a 10 minute incubation period, the epididimydes were removed and the sperm suspension incubated for a further 30 minute period. A 500 ul aliquot of the sperm suspension was removed from the top layer of media and placed in a 1.5 ml centrifuge tube. A 3 ul volume of phage combinatorial peptide library was added to this tube and the suspension incubated for 1 hour at 37 degrees. Following incubation, the sperm suspension was washed as follows. The suspension was centrifuged in a tabletop microcentrifuge at maximum speed for 1 minute and the majority of the supernatant removed. The remaining volume was re-suspended in 1 ml of media via pipetting and separated into two 500 ul aliquots in two 15 ml centrifuge tubes. A further 14 ml of CZB wash media was added to these tubes followed by 15 min incubation at room temperature. The tubes were centrifuged at 1000 g for 5 minutes and the supernatant removed down to an approximate 50 ul volume. A further 14 ml of CZB wash media was added followed by a 15 min incubation period at room temperature and identical centrifugation step. The supernatant was removed from each tube down to an approximate 50 ul volume and this remaining volume was combined with a further volume of CZB media to a final volume of 500 ul for library isolation. At each step in the protocol, an aliquot was examined and shown to contain motile sperm cells. Also, aliquots were taken from the original suspension and wash volumes for assessment of the efficiency of the screening and washing protocol. This process was repeated three times using the screened and amplified library created by prior screens. However, in the second to fourth screening protocols, a 50 ul volume of library was added to the 500 ul sperm suspension.

Combinatorial Peptide Library Handling:

The original combinatorial peptide library used in these experiments was derived from the “Ph.D” Phage Display Peptide library (New England Biolabs). Both the Ph.D-7 and Ph.D-12 libraries. General procedures and handling were done according to the protocols provided with these libraries. Bound phage were eluted at several different pH in the range of 2-5 and several concentrations in the range of 0.1 to 1 molar Tris Glycine at different times in the binding cycle. There was no panning as described in the New England Biolab protocols.

Phage DNA was isolated by several different methods. One procedure used Qiagen protocols and another procedure used AmpliTemp procedures. Both gave equivalent results. DNA sequences were obtained using two different primers as available in the New England Biolab protocols. Sequencing was done by the Columbia University Sequencing facilities on an ABI DNA sequencer using both gel and capillary flow fractionation. DNA quality and quantity was determined via agarose gel electrophoresis on vertical mini-gels with novel illumination and filters. DNA concentrations were obtained from the agarose gel by a continuous web camera with special filters on a PC computer. Sequence analysis was done using ABI software.

Peptide sequences were deduced from the phage DNA sequence using proprietary algorithms developed via the Nussinov, Pieczenik, Griggs and Kleitman algorithm homology variations (Nussinov et al, 1978) and the renormalization functions developed by Pieczenik(unpublished).

Sperm Staining

A peptide corresponding to the identified ligand sequence was directly synthesized by New England Peptide with a terminal rhodamine fluorescent label. Lyophilized peptide was originally diluted in PBS at a concentration of 2 mg/ml. A small volume of this peptide solution (3 μl) was added to 60 μl of a standard suspension of washed human spermatozoa in culture media (a˜1:20 dilution). Following a 15 minute incubation at RT, a further 60 μl volume of sperm suspension was added to this tube to dilute the peptide concentration and reduce background signal. A small volume of this suspension was used to make an air-dried smear on a standard microscope slide. When dry, a small volume of anti-fade mounting solution was placed on the slide and a coverslip mounted. Slides were imaged on an Olympus AX70 epi-fluorescent microscope. Images were captured using the Metasystems Isis 3 imaging system.

Results:

This analysis gave a binding sequence of mouse sperm with the combinatorial libraries containing the unique 6 amino acids, HIPRTY. The identification of this sequence using proprietary Database analysis revealed that the consensus sperm peptide ligand sequence is found in Axin 1 (Axis inhibition protein 1). This protein has been characterized in several species including human, rat, mouse, chick, and Xenopus.

The fluorescently-labeled peptide corresponding to the identified sperm ligand, bound to human spermatozoa (FIG. 1). The binding pattern corresponded to the posterior region of the sperm head and the flagellum. This pattern was consistent over multiple spermatozoa.

Discussion

Peptide library screening of intact, living mouse sperm revealed a robust binding ligand. This peptide sequence is a 100% match for residues of the Axin protein. These residues fall within a region identified by mutagenesis experiments in the rat as a binding site for Glycogen Synthase Kinase-3β (GSK-3β) (rat sequence AA 353-437 corresponding to the mouse sequence AA 477-561) (Ikeda et al., 1998). The residues comprising this ligand are completely conserved between the mouse, rat, chicken and human Axin sequences (Zeng et al., 1997; Ikeda et al., 1998). This area defines and extremely strong protein-protein interaction site. The interaction between Axin and GSK-3 is so robust that an affinity chromatography protocol specifically isolating GSK-3 has been reported using immobilized Axin (Primot et al., 2000). Therefore, the sperm binding ligand identified by this blind screen not only unambiguously identified a known protein, it also falls within a previously characterized, highly conserved site of protein-protein interaction with a known sperm surface protein. This result would indicate that the binding activity observed in this screen was related to GSK-3 present on the sperm. A fluorescently-labeled peptide version of this ligand bound to human spermatozoa at the posterior portion of the sperm head and along the flagellum. This pattern is essentially identical to immunohistochemical staining for GSK-3α in bovine spermatozoa as discussed below (Vijayaraghavan et al., 2000).

GSK-3 is a serine/threonine kinase known to be regulatory component in a multitude of diverse cellular signaling pathways (Wodgett, 1990; Doble and Wodgett, 2003; Jope and Johnson, 2004). GSK-3 has two common isoforms, α and β, encoded by unique genes. The two isoforms have essentially identical kinase domains and differ in an extended N-terminus region in the α isoform. Both isoforms exibit axin-binding behavior (Doble and Woodgett, 2003). GSK-3 has been characterized in the mature spermatozoa of the human, bovine, and rhesus macaque and during spermatogenesis in the mouse and rat (Vijayaraghavan et al., 1996; Smith et al., 1999; Vijayaraghavan et al., 2000; Guo et al., 2003). In the bovine, immunohistochemistry for GSK-3α shows a localization pattern in the posterior portion of the sperm head and along the flagellum (Vijayaraghavan et al., 2000). GSK-3 is believed to play a primary role in the regulation of sperm motility possibly by activation of protein phosphatase 1l (Vijayaraghavan et al., 1996).

It is unclear if this result—the unambiguous identification of a ligand protein from a vital whole cell screen—is a consistent characteristic of this library screening protocol. Prior screens using spermatozoa isolated and prepared by different methodologies did result in the convergent identification of true binding ligands. A screen of the mouse zona pellucida conducted in similar fashion and in parallel with the current study failed to identify a convergent long ligand sequence following 4 rounds of selection It did identify one unique amino acid, Thr, at the two position of a stretch of 7 amino acids (data not shown). This suggest a microheterogeneity of zona target complexity Further repetition of the identical and modified screening protocol in the spermatozoa of mouse and other species will help to clarify this question. In any case, this result demonstrates that such intact, vital, whole cell screens can identify native proteins.

Developmental indications:

The identification of Axin as a protein containing a binding ligand for mammalian sperm was initially a surprising result. The identification of this specific ligand as a site of association with a known sperm surface protein clarified this issue to some extent. It is certainly possible that the identification of sperm-associated GSK-3 via its Axin-binding behavior in this screen is coincidental. GSK-3 is a relatively ubiquitious enzyme with multiple regulatory roles and the identified ligand is an area of strong protein-protein interaction. However, an alternative concept is that the identification of the GSK-3-specific binding region of Axin as a robust sperm ligand in this screen suggests that sperm may be involved with an Axin/GSK-3β-mediated phenomenon. Since it has been proposed that mammalian sperm may play a role in embryonic axis determination; a function associated with Axin and GSK-3, this becomes an intriguing possibility.

Axin is a scaffolding protein and part of a multi-protein complex involved with the regulation of β-catenin via the Wnt signaling pathway (Zeng et al., 1997). The Axin/GSK-3 association (mediated via the binding site identified in this screen) is a critical component of this complex developmental pathway involved in determining the anterior-posterior developmental axis in various species including Xenopus and rodents (Zeng et al., 1997; Itoh et al., 1998). In the mouse, lack of Axin results in a duplication of the anterior-posterior embryonic axis due to mis-regulation of β-catenin signaling (Zeng et al., 1997). GSK-3β acts as a negative regulator in this system by phosphorylating β-catenin and thereby stimulating its degradation. The binding of extracellular ligands such as Wnt activate this system by ultimately inhibiting GSK-3β and allowing the accumulation and nuclear translocation of β-catenin which in turn activates downstream genes (Ikeda et al., 1998; Fagotto et al., 1999). The regulation of the Axin-GSK-3β complex formation and the activities of GSK-3β and other associated proteins is complicated and not fully understood. GSK-3β also phosphorylates Axin itself, apparently stabilizing the complex (Jho et al, 1999). Also, other proteins such as GSK-3-binding protein (GBP) interact with the same region of GSK-3β involved with Axin association and modulate its phosphorylation of both Axin and β-catenin (Thomas et al., 1999). The existence of tripeptide interactions is analogous to the tripeptide interactions involved in antigen recognition.

It is not clear how the actions of Wnt and downstream components are spatially regulated in the mouse embryo to bring about axis formation. The first morphological manifestation of anterior-posterior axis formation in the mouse is the generation of the primitive streak around day 6.5 of development (although existing asymmetries may underlie this process) (Smith et al., 1985; Gardner et al., 1992). Cells in the anterior end of the primitive streak may respond to signals from the adjacent extra-embryonic or visceral endoderm cells setting up axis formation mediated via the Wnt pathway. The physical and molecular events responsible for the initial generation of these developmental asymmetries remain to be determined. Analysis of Axin mutants indicates that the very earliest manifestations of axis determination are perturbed. Wnt mutants do not exhibit perturbations in the early events in axis formation and Wnt has not been shown to be expressed prior to primitive streak formation (Moon et al., 1997).

In the best studied system, Xenopus, axis determination can be traced to events generating asymmetry in the fertilized egg. Cortical rotation in the fertilized Xenopus egg relative to the sperm entry point brings about a redistribution of maternal determinants resulting in the generation of an axis determining region on what will become the ventral side of the developing embryo opposite to the sperm entry point (Kofron et al., 2001). Current evidence suggests that oriented microtubules resulting from the sperm aster are used for the kinesin-mediated transport of GBP and other proteins to this region of the cytoplasm. Asymmetrically localized GBP then mediates the localized suppression of GSK-3 activity by destabilizing the GSK-3-Axin complex leading to an increase in β-catenin in this region (Weaver et al., 2003). The cortical rotation process is apparently unique to Xenopus although evidence suggests a similar microtubule-transport-dependent process in zebrafish (Sumoy et al., 1999).

Early development in the mouse in known to be highly regulative. However, a variety of studies have suggested that asymmetries in the fertilized mouse egg and events in the developing pre-implantation embryo may at least play a role in determining the positioning of subsequent embryonic axes and the sperm entry point has been implicated in this phenomenon (Gardner 1997; Piotrowska et al., 2001). In the mouse, some studies have suggested that the sperm entry point is associated with the position of the axis of symmetry in the blastocyst and thus in subsequent downstream developmental axes (Piotrowska and Zernika-Goetz, 2001; Plusa and Zernika-Goetz, 2002). Also there is evidence that the first cleavage plane in mouse embryos does bisect the zygote passing near the sperm entry point and that the blastomere inheriting the sperm entry point exhibits more rapid second cleavage (Pitrowska and Zernicka-Goetz, 2001). However, the role of sperm entry in determining early and downstream asymmetries has been strongly debated and it is possible that such asymmetries are simply pre-existing in the oocyte (Gardner and Davies, 2003; Zernika-Goetz, 2003; Johnson, 2003). Evidence from a variety of studies demonstrates that the animal-vegetal axis of the egg and polar body position can be traced forward to the asymmetric positioning of cells within the blastocyst inner cell mass (ICM) and regions of the visceral endoderm that will define the anterior-posterior embryonic axis (Gardner 1997; Weber et al., 1999; Beddington and Robertson, 1999; Ciemerych et al., 2000; Piotrowska et al., 2001). The recent birth of mice from true oocyte parthenogenesis provides final proof that spermatozoa (and positional information derived from the sperm entry point) are not absolutely required for the generation of downstream asymmetries and correct mammalian development (Kono et al., 2004). However, this does not rule out the possibility that such sperm dependant positional information may have a dispensable role, or ancestral role, or may be the actual mechanism used in a context of redundant vestigal systems. Duplication of function with alternate structures is a common evolutionary protective mechanism.

Our finding of a component of the Axin/Wnt pathway associated with sperm, provides the first specific potential molecular basis of a role for sperm in the generation of early asymmetries. GSK-3 associated with the internalized spermatozoa could bind to Axin, GBP, and/or other components of this system creating an asymmetric distribution of a unique protein complex in the fertilized egg and in downstream blastomeres and embryonic regions. While a study examining the distribution of directly-labeled sperm components during early cleavage failed to discern any relevant asymmetries, this does not rule out the concept that transient interaction with the spermatozoa could not set up such asymmetries in cytoplasmic components (Gardner and Davies, 2003). A sperm-associated protein has not been directly implicated in axis formation in any species and it is difficult to envision a mammalian role for Axin-GSK-3 similar to the situation in Xenopus. Furthermore, evidence indicates that a Wnt gradient acting on an essentially uniform Axin-GSK-3 distribution determines the subsequent β-catenin gradient and axis development in the day E6.5 mouse embryo. However, the Axin-GSK-3 complex is known to be subject to a variety of subtle regulative controls and these may be involved with setting up and “fine-tuning” of this process. Upstream asymmetries derived from sperm-associated GSK-3 could conceivably play at least a dispensable role in this regulation. It is also possible that sperm-associated GSK-3 may be involved with mediating other cryptic events in early development (unrelated to axis orientation perse) through an association with cytoplasmic Axin, GBP or other proteins.

Due to the regulatory nature of early development in the mouse, the clearly dispensable role of sperm in this process, and the prevalence of GSK-3 in other cellular processes it is difficult to conceive of experimental manipulations that might reveal a role for sperm-associated GSK-3 in development. The labeling experiments discussed above have demonstrated a connection between specific cellular distributions in post-implantation embryos and earlier physical asymmetries such as the egg axis and first cleavage planes. Possibly sperm or early-cleavage-specific interference with GSK-3 function (or Axin binding) might be applied in combination with labeling studies to reveal a potential effect. Our synthesized peptide ligand preparation might be a useful reagent in such studies. This peptide might also have an effect via GSK-3 on sperm function (motility, binding behavior, etc) and studies to examine this phenomenon are currently ongoing.

The peptides HIPRT and HIPRTY can be used to diagnose sperm for fertility issues and also to sort sperm by their ability to bind this sequence. These peptides can be used therapeutically to either inhibit or enhance sperm motility depending on extensions to these sequences.

What is claimed is:

1. A peptide comprising the sequence HIPRT.

2. A lead compound comprising the sequence HIPRT.

3. The method of using the peptide of claim 1 for therapy.

4. The method of claim 3 with the additional step of using the peptide of claim 1 for diagnosis.
